(19) United States
(12) Patent Application Publication
(10) Pub. No.: US 2025/0384964 A1
(43) Pub. Date: Dec. 18, 2025

(54) METHOD FOR ANALYZING TARGET ORGANISM

(71) Applicant: NIHON BIODATA CORPORATION, Kanagawa (JP)

(72) Inventor: Norichika OGATA, Kanagawa (JP)

(73) Assignee: NIHON BIODATA CORPORATION, Kanagawa (JP)

(21) Appl. No.: 18/877,272

(22) PCT Filed: Aug. 8, 2023
(86) PCT No.: PCT/JP2023/029001

§ 371 Date: Dec. 20, 2024

(30) Foreign Application Priority Data
Aug. 8, 2022 (JP) 2022-126428

(51) Int. Cl. G16B 50/50; G16B 45/00

(52) U.S. Cl. CPC G16B 50/50; G16B 45/00

(57) ABSTRACT

[Problem] To provide a method for analyzing a target organism, which can analyze data from a compression ratio without normalization, and a method for acquiring a graph from which various types of biological information can be analyzed. [Solution] A method for analyzing a target organism, comprising compression ratio fluctuation examination step of obtaining a compression ratio of sequence data on the target organism for each variable related to the target organism, wherein the compression ratio fluctuation examination step includes: a sequence data acquisition step of obtaining a plurality of pieces of sequence data based on the target organism for each of the variables; and a compression ratio calculation step of compressing the plurality of pieces of sequence data to obtain a data compression ratio.

### TECHNICAL FIELD

The present invention relates to a method for analyzing a target organism.

### BACKGROUND ART

Japanese Patent No. 6872744 describes a method for normalizing transcriptome data. Japanese Patent No. 6979280 describes a method for examining transcriptome data. Japanese Patent No. 6342533 describes a method for extracting a differentially expressed gene using transcriptome or selecting an experimental group targeted for pathway analysis. The method described in Japanese Patent No. 6979280 measures the file sizes of a plurality of pieces of compressed transcriptome data after a size unifying step. In the size unifying step, each piece of data included in the plurality of pieces of transcriptome data is converted into a binary digit, and the size of each piece of data is unified by making the digit number of the converted binary bit data uniform. As described above, normalization is essential for transcriptome data examination.

CITATION LIST

### Patent Literature

Patent Literature 1: Japanese Patent No. 6872744.

Patent Literature 2: Japanese Patent No. 6979280.

Patent Literature 3: Japanese Patent No. 6342533.

### SUMMARY OF INVENTION

### Technical Problem

Provided is a method for analyzing a target organism, which can analyze data from a compression ratio without normalization, and a method for acquiring a graph from which various types of biological information can be analyzed.

### Solution to Problem

The present invention is basically based on a finding that sequence data on a target organism is obtained, the compression ratio of the obtained sequence data is obtained, and the target organism can be analyzed accordingly.

One aspect of the invention relates to a method for analyzing a target organism. This method is preferably a method implemented by a computer.

The method includes a compression ratio fluctuation examination step of obtaining the compression ratio of sequence data on the target organism for each variable related to the target organism.

The compression ratio fluctuation examination step includes:

a sequence data acquisition step of obtaining a plurality of pieces of sequence data based on the target organism for each of the variables; and

a compression ratio calculation step of compressing the plurality of pieces of sequence data to obtain a data compression ratio.

The compression ratio calculation step may include a sequence data compression step of obtaining compressed sequence data.

Examples of the variable include one type or two or more types of variables related to metadata on the target organism, the number of days of cultivation, the amount of specific substance to be administered to the target organism, the number of times of administration of the specific substance to the target organism, and a cultivation environment for the target organism.

Examples of the sequence data include base sequence data.

Examples of the base sequence data include:
(1) fastq base sequence data; and
(2) base sequence data obtained by removing any one or both of an ID line and a QV line from fastq base sequence data.

The plurality of pieces of sequence data based on the target organism may be:
(1) data on a base sequence in the target organism; or
(2) data on a base sequence in the target organism under a cultivation environment.

The method for analyzing the target organism may further include a graph creation step of creating a graph taking the variable as a first axis and the compression ratio as a second axis.

Preferred examples of the method for analyzing the target organism include causing a computer to execute each step.

One aspect of the invention relates to a program. The program is a program causing the computer to execute any of the methods described above.

One aspect of the invention relates to an information recording medium. The information recording medium is a computer-readable non-transitory information recording medium storing the above-described program.

### Advantageous Effects of Invention

According to the method, the method for analyzing the target organism can be provided, which can analyze data without normalization.

### BRIEF DESCRIPTION OF DRAWINGS

FIG. 1 is a graph provided as a diagram showing a relationship between metadata (distance from a tip end of a plant body) of base sequence data and a compression ratio obtained in Example 1.

FIG. 2 is a graph provided as a diagram showing a relationship between the metadata (distance from the tip end of the plant body) of the base sequence data and an information entropy obtained in Reference Example 1.

FIG. 3 is a graph provided as a diagram showing a relationship between the metadata (distance from the tip end of the plant body) of the base sequence data and the compression ratio of fastq base sequence data.

FIG. 4 is a diagram showing a relationship between the compression ratio of base sequence data obtained by removing an ID line and a QV value from the fastq obtained in Example 3 and the information entropy of gene expression quantitative data.

FIG. 5 is a graph provided as a diagram showing a relationship between the compression ratio obtained in Example 2 and the information entropy obtained in Reference Example 1.

FIG. 6 is a graph provided as a diagram showing a relationship between the compression ratio and the date and time of cultivation for analyzing the degree of cell maturation.

FIG. 7 is a graph provided as a diagram showing a relationship between the compression ratio and a cultivation time for grasping the progress of microorganism maturation in the course of cultivation.

FIG. 8 is a graph provided as a diagram showing a relationship between the compression ratio and a medical agent concentration in a culture medium when a medical agent is added to the culture medium in which an animal cell is cultivated, for examining a gene responding to the medical agent.

### DESCRIPTION OF EMBODIMENTS

Hereinafter, embodiments of the present invention will be described with reference to the drawings. The present invention is not limited to the embodiments described below, and also includes modifications made as necessary within a scope obvious to those skilled in the art from the embodiments below.

One aspect of the invention relates to a method for analyzing a target organism. Examples of the target organism include arbitrary plants and arbitrary animals. The target organism preferably includes plants and agricultural crops. Another aspect of the method relates to a method for classifying base sequence data without mapping (without normalization or the like). The method for analyzing the target organism may be a method for examining transcriptome data for analyzing biological significance.

The method is preferably a method implemented by a computer. The computer has input and output units, a storage unit, a control unit, and an arithmetic unit, and each element is capable of transmitting and receiving information via a bus or the like. The computer is only required to read a control program stored in the storage unit and perform various types of arithmetic processing. The computer may be connected to a server via the Internet or the like, and the server may store various types of data and perform a predetermined type of arithmetic processing. In a case where a predetermined type of information is input from the input unit, the control unit reads the control program stored in the storage unit. Then, the control unit reads information stored in the storage unit as necessary, and transmits such information to the arithmetic unit. Moreover, the control unit transmits input information to the arithmetic unit as necessary. The arithmetic unit performs arithmetic processing using various types of information received, and stores an arithmetic processing result in the storage unit. The control unit reads the arithmetic processing result stored in the storage unit, and outputs such a result from the output unit. In this manner, various types of processing and each step are executed. The computer may include a processor and a memory coupled to the processor. The memory stores a command, and the command may cause, when executed by the processor, the computer to perform various steps or to function as various elements. The computer may build a learning model using various types of training data and implement various types of arithmetic processing by machine learning. In this case, the computer may execute various types of examination and analysis using a learning model created by machine/deep learning of artificial intelligence (AI).

The analyzing the target organism includes any type of analysis. Preferred examples of the analyzing the target organism include classifying the target organism. For example, the target organism is classified according to whether or not harvest timing has come, at what point in time it is before harvesting, or the like. The analyzing the target organism includes determining whether or not the target organism is in a preset state. Further, the analyzing the target organism includes obtaining, when a certain organism is classified, a correlation between known useful data and a compression ratio and analyzing whether or not the compression ratio is available as a substitute for the useful data or performing examination using the compression ratio instead of the useful data.

The method includes a compression ratio fluctuation examination step of obtaining the compression ratio of the sequence data on the target organism for each variable related to the target organism. The compression ratio fluctuation examination step includes a sequence data acquisition step and a compression ratio calculation step.

### Sequence Data Acquisition Step

The sequence data acquisition step is a step of obtaining, for each variable, a plurality of pieces of sequence data based on the target organism. A sample of the target organism is collected, and the plurality of pieces of sequence data based on the target organism can be obtained using a well-known sequencer. The sequence data to be obtained normally includes a plurality of types of amino acid and base (DNA, RNA, or the like) derived from various cells. These amino acids and bases normally have different sequences, and have a plurality of types of residue number and base number.

Examples of the variable include one type or two or more types of variables related to metadata on the target organism, the number of days of cultivation, the amount of specific substance to be administered to the target organism, the number of times of administration of the specific substance to the target organism, and a cultivation environment for the target organism. Under different conditions, the cell of the target organism is collected, and the amino acid residue number and base sequence thereof can be obtained using the sequencer. The metadata on the target organism is data related to data on the target organism. In a case where target data is the base sequence data, examples of the metadata on the target organism include organism species as an experimental condition associated with the target data, information on the sequencer, a method for obtaining the sequence data, and the like, and the data can be classified and organized based on the metadata. Other examples of the metadata include a distance from a certain region. For example, in a case where the variable is the number of days of cultivation, the value of the variable is a value such as the start of cultivation, the first day of cultivation, the second day of cultivation, the third day of cultivation, . . . Examples of the amount of specific substance to be administered to the target organism include the amount of medicine to be administered to a patient and the amount of fertilizer to be administered to a plant. Examples of the variable related to the cultivation environment for the target organism include the amount of specific substance to be added to a culture medium, a cultivation temperature, a solar radiation time, and a humidity.

Examples of the plurality of pieces of sequence data based on the target organism include an amino acid residue number and a base sequence (DNA or RNA).

Examples of the sequence data include base sequence data.

Examples of the base sequence data includes:
(1) fastq base sequence data; and
(2) base sequence data obtained by removing any one or both of an ID line and a QV line from fastq base sequence data.

The FASTQ format is a text-based format, and is used when the base sequence of DNA or the like and the quality score thereof are saved together as one file. Each of the base sequence and the quality score is represented by one ASCII code, and therefore, a correspondence relationship between the base and the quality is easily understandable.

In the FASTQ file, one sequence is described using four lines. The first line starts with an at sign, followed by the ID of the sequence and optional description. The second line describes the base sequence. The third line describes a character “+.” In some cases, the ID of the sequence is described thereafter. The fourth line describes the quality value of the sequence described in the second line. The quality value has the same number of characters as that of the sequence in the second line.

Here, the quality value in the second or fourth line is preferably used. There are various methods for expressing the base sequence. In the method of the present invention, the fastq base sequence data is preferably used. With the fastq base sequence data, sequence information is easily extracted, and the compression ratio is easily checked or the like because of the presence of the quality value.

The plurality of pieces of sequence data based on the target organism may be:
(1) data on the base sequence in the target organism; or
(2) data on the base sequence in the target organism under the cultivation environment.

The data on the base sequence in the target organism is, for example, information on any type of base in a cell of a plant on the first day of cultivation. The sequence data may indicate the base sequence derived from the cell in the target organism itself, as described above.

The data on the base sequence in the target organism under the cultivation environment may be, for example, the base sequence of a cell under an environment where a substance derived from the target organism, such as culture supernatant, is included. For example, in order to check/classify the degree of fermentation or to check/classify the progress of fermentation of a fermented product (fermented food (for example, fermented milk, sake, soy sauce, and miso), compost, or the like), the base sequence of the cell in the target organism under the cultivation environment (fermented milk, sake, soy sauce, miso, compost, or the like) may be obtained.

After the cell targeted for the analysis and the like have been obtained, the sequence data can be obtained using the well-known sequencer. The sequence data obtained as described above is input to the system. The system can obtain the plurality of pieces of sequence data in this manner. Note that at this time, the information on the value of the variable is also preferably input to the system. Moreover, the system preferably stores, in the storage unit, the plurality of pieces of sequence data input in association with the value of the variable.

### Compression Ratio Calculation Step

The compression ratio calculation step is a step of compressing the plurality of pieces of sequence data obtained in the sequence data acquisition step to obtain the data compression ratio. If all the plurality of pieces of sequence data are the same sequence data, a compression efficiency is extremely high. However, the sequence data targeted for compression in the compression ratio calculation step is not normalized by mapping or the like, and for this reason, normally includes not only different pieces of sequence data but also different sequence listings. The system compresses each of the plurality of pieces of sequence data obtained in the sequence data acquisition step. At this time, in a case where the sequence data includes data (ID or the like) other than the amino acid residue number and the base sequence data, these pieces of data are deleted and the sequence data is then compressed. Normally, the ID or the like is tagged, or the contents thereof are specified by a line number, and for this reason, the data other than the sequence can be easily deleted. The data to be deleted at this time, such as the ID or the organism species, may also be used as the metadata.

When the plurality of pieces of sequence data targeted for compression are obtained, the system stores such sequence data as necessary. At this time, the system may store each piece of the sequence data with identification information. Then, the system measures the file size of the file including each piece of the sequence data, and obtains a file capacity (file size) before compression. Examples of the file size include 20 kilobytes (kb). The file size can be easily obtained, for example, using a UNIX (registered trademark) Is program. The system stores, in the storage unit, the file size of each piece of sequence data together with the identification information thereon.

Next, the plurality of pieces of sequence data are compressed, and compressed sequence data is obtained. Examples of a compression method include a zip method, a tar method, a gzip method, an LZH method, a bzip2 method, a tbz method, a tar.xz method, a 7-zip method, a rar method, a taz method, a SIT method, a GCA method, a CAB method, a SEA method, a HQX method, a BIN method, an IMG method, a SMI method, a CPT method, a compress(z) method, an ARJ method, and a cab method. In order to compress the sequence data, for example, a UNIX (registered trademark) zip program may be used. For example, by compressing each piece of sequence data using the UNIX (registered trademark) zip program, the compressed sequence data can be obtained. The system reads the sequence data from the storage unit, and compresses the read sequence data based on a compression program instruction to obtain the compressed sequence data. The obtained compressed sequence data may be stored in the storage unit in association with the identification information.

Next, the file size of each piece of compressed sequence data is measured, and the file capacity (file size) after compression is obtained. The system reads the compressed sequence data from the storage unit, and obtains the file size of the compressed sequence data based on a program instruction. Examples of the file size include 5 kilobytes (kb). The file size can be easily obtained, for example, using the UNIX (registered trademark) Is program. The system stores the obtained file size of the compressed sequence data in the storage unit, as necessary. In some cases, the sequence data is input in a compressed state to the system. In this case, the system may store the compressed file in the storage unit as necessary, decompress the compressed file in response to a decompression program instruction, and store the decompressed file in the storage unit as necessary. Thereafter, the system may acquire the file size before compression.

Thereafter, the system obtains the compression ratio. The system reads, according to the identification information, the file size of the pre-compressed sequence data and the file size of the compressed sequence data, and causes the arithmetic unit to perform arithmetic processing of obtaining the ratio therebetween. The system may cause the arithmetic unit to perform arithmetic processing of obtaining an average for the plurality of pieces of sequence data. In this manner, the system can obtain the compression ratio. Examples of the compression ratio include 0.25. The system stores the obtained compression ratio in association with the value of the variable, as necessary.

The method for analyzing the target organism may further include a graph creation step of creating a graph taking the variable as a first axis and the compression ratio as a second axis. For example, the graph may be created with the metadata (here, distance from a tip end of a plant body) of the base sequence data as the horizontal axis and the compression ratio (the ratio of the electronic data file size before and after compression) as the vertical axis. With the graph, a relationship between the value of the variable and the compression ratio is obvious at a glance. The system may read the value of each variable stored in the storage unit and the value of the compression ratio corresponding to such a value of the variable, and produce the graph based on an instruction from a program for creating the graph. The value of the variable and a color corresponding to the value of the variable may be stored in the storage unit, and a colored graph may be created. In a case where there is a point indicating an abnormal value or a non-grouped point on the graph, the abnormal value point or the like may be displayed in a color different from those of other points. The created graph may be stored in the storage unit as necessary so that the graph can be output. This graph taking the variable as the first axis and the compression ratio as the second axis is extremely effective for analyzing (classifying or determining) the target organism. For example, in a case where fermentation has been completed, the compression ratio is assumed to be high because the type of substance in the environment system is unified. With the information on the value of the variable (the number of days of cultivation, the amount of substance added, the cultivation temperature, or the like) corresponding to the high compression ratio, various types of information such as at what timing fermentation is completed, how much the fertilizer needs to be administered, or at what temperature cultivation needs to be made can be obtained. A predetermined compression ratio is stored in advance, and when the compression ratio reaches such a predetermined compression ratio, the organism can be classified as, for example, fermentation completed (the organism brought into a harvestable state). For example, the data sizes are obtained for a group administered with a certain sample of 1 mg, a group administered with a sample of 10 mg, a group administered with a sample of 1 mg once a day, a group administered with a sample of 1 mg three times a day, and a group administered with a sample of 5 mg three times a day, so that the most suitable administration amount and frequency can be easily grasped.

Preferred examples of the method for analyzing the target organism include causing the computer to execute each step.

One aspect of the invention relates to a program. The program is a program causing the computer to execute any of the methods described above.

One aspect of the invention relates to an information recording medium. The information recording medium is a computer-readable non-transitory information recording medium storing the above-described program. Examples of the information recording medium include a CD, a CD-ROM, a DVD, a USB memory, a hard disc, a SD card, and a Blu-ray Disc.

### Example 1

The present invention will be specifically described with reference to examples. The present invention is not limited to the examples below.

From DDBJ SRA, base sequence data DRR187484, DRR187485, DRR187486, DRR187487, DRR187488, DRR187489, DRR187490, DRR187491, DRR187492, DRR187493, DRR187494, DRR187496, DRR187497, DRR187498, DRR187499, DRR187500, DRR187501, DRR187502, DRR187503, DRR187504, DRR187505, DRR187506, DRR187507, DRR187508, DRR187511, DRR187512, DRR187513, DRR187514, DRR187515, DRR187516, DRR187517, DRR187518, DRR187519, DRR187520, DRR187521, DRR187522, DRR187523, DRR187526, and DRR187527 was acquired. From each piece of the base sequence data, ten thousand lines of the base sequence data were extracted, and electronic data was saved as a file.

The file size of each piece of electronic data was obtained using the UNIX (registered trademark) Is program.

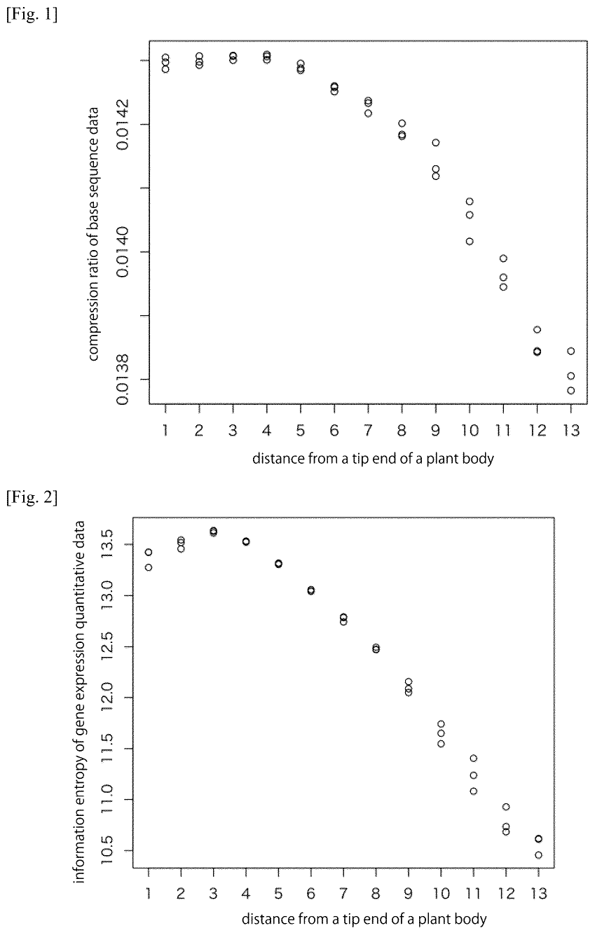

Next, each piece of electronic data was compressed using the UNIX (registered trademark) zip program. The file size of each piece of compressed electronic data was obtained using the UNIX (registered trademark) Is program. The ratio of the file size of the electronic data before and after compression was taken as the compression ratio, and the compression ratio and the metadata (here, distance from the tip end of the plant body) of the base sequence data were plotted. Results are shown in FIG. 1.

FIG. 1 is a graph provided as a diagram showing a relationship between the metadata (distance from the tip end of the plant body) of the base sequence data and the compression ratio obtained in Example 1. FIG. 1 shows data groups corresponding to the compression ratios according to the distance from the tip end of the plant body. In other words, the data corresponding to a predetermined range of the compression ratio can be classified as falling within a predetermined range in terms of the distance from the tip end of the plant body. As the distance from the tip end of the plant body increases, differentiation progresses and various tissues are formed. In a case where the sequence (amino acid sequence or base sequence) in a cell is acquired in descending order of the distance from the tip end of the plant body, various types of sequence are assumed to be obtained. When the sequence varies as described above, the compression efficiency is assumed to be low. The results of FIG. 1 show that such biological assumption is well consistent with the grouping according to the compression ratio.

### Reference Example 1

The same base sequence data as that of Example 1 was mapped on wheat genome data (iwgsc_refseqv2.1_assembly.fa), and gene expression quantitative data was created for each specimen. An information entropy was obtained from each piece of gene expression quantitative data. The metadata of the base sequence data was plotted together with the compression ratio obtained in Example 1. A graph obtained in this manner is shown in FIG. 2.

FIG. 2 is a graph provided as a diagram showing a relationship between the metadata (distance from the tip end of the plant body) of the base sequence data and the information entropy obtained in Reference Example 1. As a result of comparison between the compression ratio obtained in Example 1 and the information entropy obtained in Reference Example 1, there was a correlation therebetween. In Reference Example 1, mapping was performed, and therefore, the transcriptome data can be classified as in Patent Literatures 1 to 3. As a result of comparison of FIG. 2 with FIG. 1, classification similar to that in the case of performing mapping was made in Example 1 although mapping or normalization was not performed. This means that the present invention can classify the biological data without mapping.

### Example 2

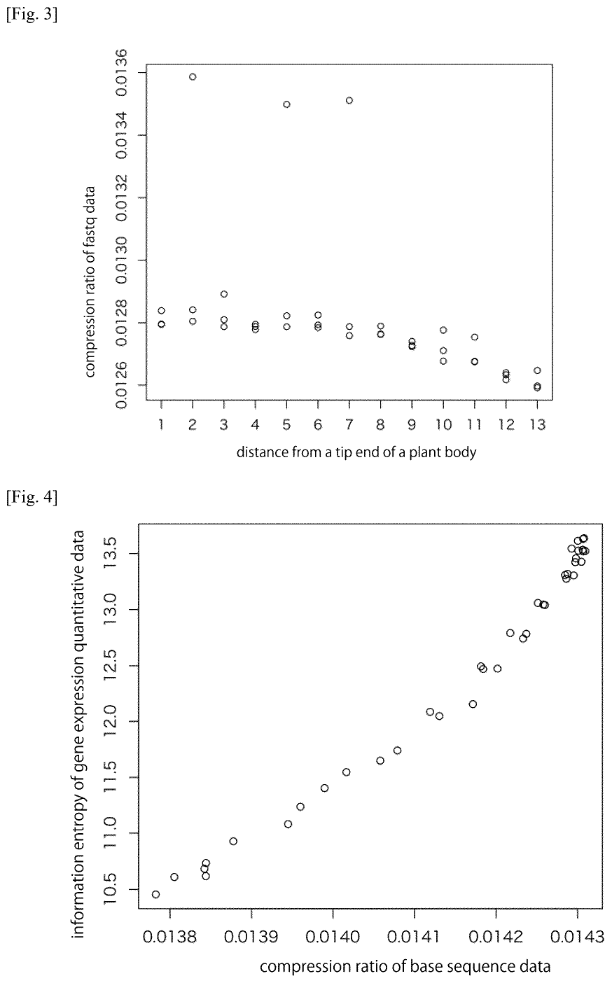

From each piece of the same base sequence data as that in Example 1, ten thousand lines of the base sequence data were extracted, and electronic data was saved as a file. The file size of each piece of electronic data was obtained using the UNIX (registered trademark) Is program. Each piece of electronic data was compressed using the UNIX (registered trademark) zip program. The file size of each piece of compressed electronic data was obtained using the UNIX (registered trademark) Is program. The ratio of the file size of the electronic data before and after compression was taken as the compression ratio, and the compression ratio and the metadata of the base sequence data were plotted. The obtained graph is shown in FIG. 3.

FIG. 3 is a graph provided as a diagram showing a relationship between the metadata (distance from the tip end of the plant body) of the base sequence data and the compression ratio of the fastq base sequence data.

### Example 3

The compression ratio of the base sequence data obtained by removing the ID line and the QV value from the fastq data can be used instead of the information entropy of the gene expression quantitative data. The compression ratio was obtained in a manner similar to that of Example 1, except that the base sequence data obtained by removing the ID line and the QV value from the fastq data was used. According to the methods described in Patent Literatures 1 to 3, the information entropy of the gene expression quantitative data was obtained. Results are shown in FIG. 4.

FIG. 4 is a diagram showing a relationship between the compression ratio of the base sequence data obtained by removing the ID line and the QV value from the fastq obtained in Example 3 and the information entropy of the gene expression quantitative data.

### Example 4

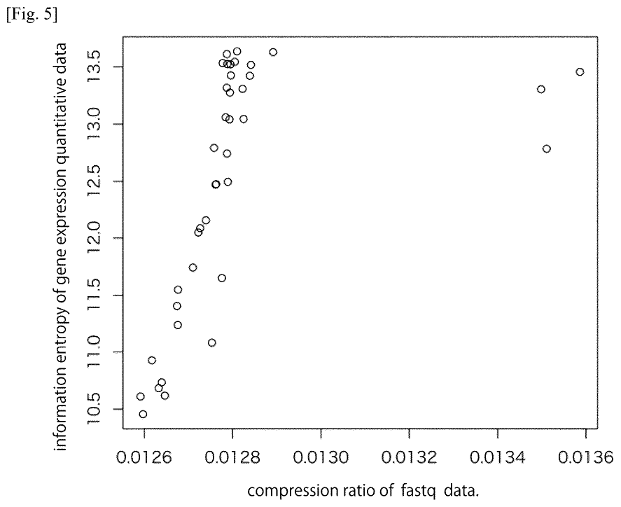

The compression ratio obtained in Example 2 and the information entropy obtained in Reference Example 1 were compared. Results are shown in FIG. 5.

FIG. 5 is a graph provided as a diagram showing a relationship between the compression ratio obtained in Example 2 and the information entropy obtained in Reference Example 1. As shown in FIG. 5, there were outliers, but there was a correlation between the compression ratio and the information entropy. This shows that the compression ratio of the fastq data can be used instead of the information entropy of the gene expression quantitative data. Particularly, it is assumed that in a case where the data obtained from the same run of the sequencer has QV values close to each other, the compression ratio of the fastq data is suitably used instead of the information entropy of the gene expression quantitative data. Note that FIG. 5 shows points indicating abnormal values and non-grouped points. The abnormal value points or the like may be displayed in a color different from those of other points.

### Consideration

In the above-described examples, the base sequence of the plant was obtained for each distance from the tip end of the plant, and these base sequences were examined.

However, the present invention is not limited to these examples.

### Example 5

When an animal cell is cultivated using an incubator for the purpose of cell production, cell maturation is insufficient in a case where harvesting is extremely earlier than ideal timing, and cell maturation is sufficient, but the occupation time of the incubator increases and cost performance is lowered in a case where harvesting is extremely later than the ideal timing. In order to quantitatively grasp a cell maturation state, messenger RNA derived from a cell was extracted, and sequencing was performed using a next-generation sequencer manufactured by Illumina, Inc. In this manner, the base sequence data was acquired. The base sequence data was zip compressed, and was copied to a calculator. The file size of the zip-compressed base sequence data was recorded, and thereafter, the zip-compressed base sequence data was decompressed and the file sizes of the base sequence data before and after compression were recorded. The file sizes of the base sequence data before and after compression were compared, and the compression ratio of the base sequence data was recorded. The compression ratio of the base sequence data on each biospecimen and the number of days of cultivation for the culture derived from each biospecimen were plotted so that the progress of cell maturation in the course of cultivation can be grasped.

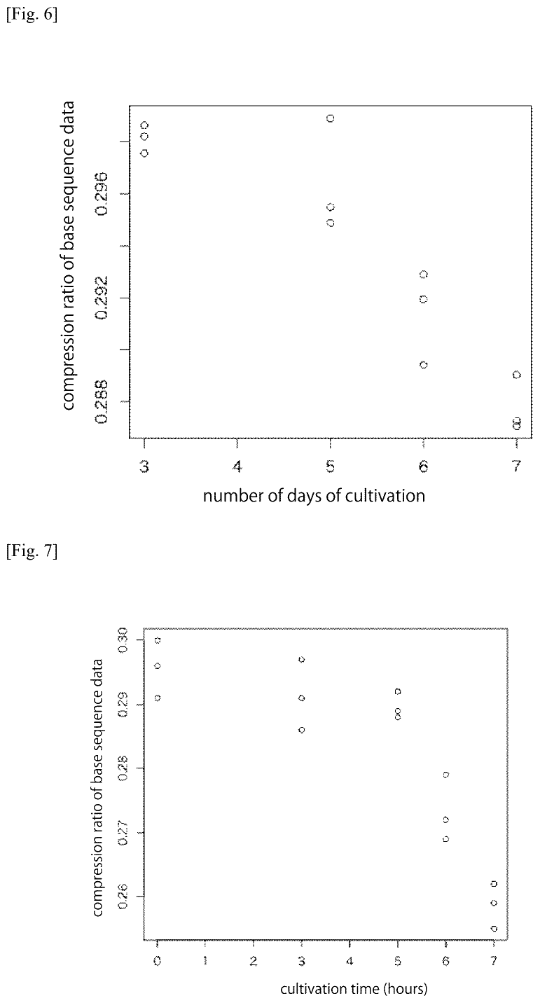

FIG. 6 is a graph provided as a diagram showing a relationship between the compression ratio and the date and time of cultivation for analyzing the degree of cell maturation. As shown in FIG. 6, it is grasped from the graph that cell maturation was sufficient on the sixth day of cultivation. Thus, the cell was harvested on the sixth day of cultivation.

### Example 6

When a microorganism is cultivated using an incubator for the purpose of microbial cell production, microorganism maturation is insufficient in a case where harvesting is extremely earlier than ideal timing, and microorganism maturation is sufficient, but the occupation time of the incubator increases and cost performance is lowered in a case where harvesting is extremely later than the ideal timing. In order to quantitatively grasp a microorganism maturation state, messenger RNA derived from a microorganism was extracted, and sequencing was performed using the next-generation sequencer manufactured by Illumina, Inc. In this manner, the base sequence data was acquired. The base sequence data was zip compressed, and was copied to the calculator. The file size of the zip-compressed base sequence data was recorded, and thereafter, the zip-compressed base sequence data was decompressed and the file sizes of the base sequence data before and after compression were recorded. The file sizes of the base sequence data before and after compression were compared, and the compression ratio of the base sequence data was recorded. The compression ratio of the base sequence data on each biospecimen and the number of days of cultivation for the culture derived from each biospecimen were plotted so that the progress of microorganism maturation in the course of cultivation can be grasped.

FIG. 7 is a graph provided as a diagram showing a relationship between the compression ratio and a cultivation time for grasping the progress of microorganism maturation in the course of cultivation. As shown in FIG. 7, it is grasped that microorganism maturation was sufficient after a lapse of six hours of cultivation. Thus, the cell was harvested after a lapse of six hours of cultivation.

### Example 7

In order to examine a gene responding a medical agent, experiment is conducted, in which the medical agent is added to a culture medium in which an animal cell is cultivated and the cell cultivated in the culture medium is examined. However, an ideal medical agent concentration in the culture medium is unknown. The ideal medical agent concentration in the culture medium is assumed to be a medical agent concentration which is not too high to such an extent that the transcriptome of the cell changes the behavior of the cell cultivated in the culture medium containing no medical agent at all and is not too low to such an extent that no response to the medical agent is observed. In order to quantitatively grasp the state of the transcriptome of the cell, messenger RNA derived from the cell was extracted, and sequencing was performed using the next-generation sequencer manufactured by Illumina, Inc. In this manner, the base sequence data was acquired. The file size of the base sequence data was recorded. Thereafter, the base sequence data was LZW compressed, and the file size of the compressed base sequence data was recorded. The file sizes of the base sequence data before and after compression were compared, and the compression ratio of the base sequence data was recorded. The compression ratio of the base sequence data on each biospecimen and the medical agent concentration of the culture, which is derived from each biospecimen, in the culture medium were plotted so that the response of the transcriptome along with an increase in the medical agent concentration can be grasped.

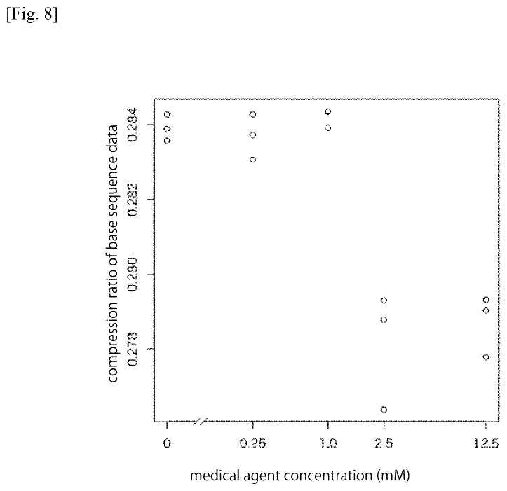

FIG. 8 is a graph provided as a diagram showing a relationship between the compression ratio and the medical agent concentration in the culture medium when the medical agent is added to the culture medium in which the animal cell is cultivated, for examining the gene responding to the medical agent. FIG. 8 shows that when the medical agent concentration exceeds 1 mM, the state of the transcriptome of the cell greatly changed. Thus, gene expression of the cell in the culture having a medical agent concentration of 0 mM in the culture medium and gene expression of the cell in the culture having a medical agent concentration of 1 mM in the culture medium were compared.

### Industrial Applicability

The present invention is useful in the information analysis industry and the pharmaceutical industry.

1. A method for examining transcriptome data on a target organism, comprising
a compression ratio fluctuation examination step of obtaining a compression ratio of sequence data on the target organism for each variable related to the target organism, wherein
the compression ratio fluctuation examination step includes:
a sequence data acquisition step of obtaining a plurality of pieces of sequence data based on the target organism for each of the variables; and
a compression ratio calculation step of compressing the plurality of pieces of sequence data to obtain a data compression ratio.

2. The method according to claim 1, further comprising
a graph creation step of creating a graph taking the variable as a first axis and the compression ratio as a second axis.

3. The method according to claim 1, wherein
the variable is one type or two or more types of variables related to the number of days of cultivation, an amount of specific substance to be administered to the target organism, the number of times of administration of a specific substance to the target organism, and a cultivation environment for the target organism.

4. The method according to claim 1, wherein
the sequence data is base sequence data.

5. The method according to claim 1, wherein
the sequence data is:
(1) fastq base sequence data; or
(2) base sequence data obtained by removing any one or both of an ID line and a QV line from fastq base sequence data.

6. The method according to claim 1, wherein
the plurality of pieces of sequence data based on the target organism are:
(1) data on a base sequence in the target organism; or
(2) data on a base sequence in the target organism under a cultivation environment.

7. A method for examining transcriptome data on a target organism using a computer, the method comprising
a compression ratio fluctuation examination step of obtaining a compression ratio of sequence data on the target organism for each variable related to the target organism by the computer, wherein
the compression ratio fluctuation examination step includes:
a sequence data acquisition step of obtaining a plurality of pieces of sequence data based on the target organism for each of the variables by the computer; and
a compression ratio calculation step of compressing the plurality of pieces of sequence data to obtain a data compression ratio by the computer.

8. A program causing the computer to execute the method according to claim 7.

9. A computer-readable non-transitory information recording medium storing the program according to claim 8.

10. The method according to claim 1, wherein
the variable is one type or two or more types of a distance from a tip end of a plant body that is the target organism, a medical agent concentration in a culture medium in which the target organism is cultivated when a medical agent is added to the culture medium, an amount of medicine to be administered to a patient that is the target organism, and an amount of fertilizer to be administered to a plant that is the target organism.

11. The method according to claim 1, wherein
the plurality of pieces of sequence data based on the target organism are transcriptome data on the target organism.
